(19) United States
(12) Patent Application Publication
(10) Pub. No.: US 2022/0242810 A1
(43) Pub. Date: Aug. 4, 2022

(54) CRYSTALLINE CANNABIGEROL

(71) Applicant: Purisys LLC, Athens, GA (US)

(72) Inventors: Wen-Chun Zhang, Bogart, GA (US); Phillip Wilkerson, Athens, GA (US); Gnel Mkrtchyan, Watkinsville, GA (US)

(21) Appl. No.: 17/596,537

(22) PCT Filed: Jun. 12, 2020
(86) PCT No.: PCT/US2020/037570

§ 371 Date: Dec. 13, 2021

Related U.S. Application Data
(60) Provisional application No. 62/861,520, filed on Jun. 14, 2019.

(51) Int. Cl. C07C 39/19

(52) U.S. Cl. CPC C07C 39/19; C07B 2200/13

(57) ABSTRACT

The application relates to crystalline cannabigerol comprising at least one X-ray powder diffraction peak selected from the group consisting of 4.73°, 9.52°, 14.30°, and 23.93° 2Θ(each ±0.20° 2Θ), to methods of making the crystalline cannabigerol and its medical uses.

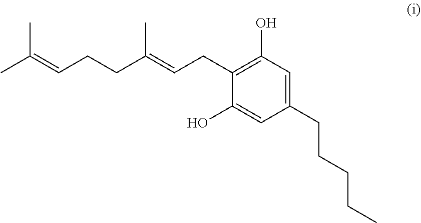 [SMILES: CCCCCc1cc(O)c(C/C=C(\C)CCC=C(C)C)c(O)c1]

### CROSS-REFERENCE OF RELATED APPLICATIONS

This application claims the benefit of priority to U.S. Provisional Patent Application No. 62/861,520, filed on Jun. 14, 2019, the content of which is incorporated by reference herein in its entirety for all purposes.

### FIELD

The subject matter described herein relates to new synthetic routes for the preparation of crystalline cannabigerol and compositions thereof.

### BACKGROUND

The characterization and selection of a solid compound for use in a pharmaceutical composition is a complex process, as even the slightest modifications in the solid's form may affect the compound's physical and chemical properties. These properties may offer potential drawbacks or advantages influencing the composition's pharmaceutical characteristics, such as processing, formulation, stability, bioavailability, storage, and handling. Crystalline solids and amorphous solids are used as solids in pharmaceutical compositions, with the product type and mode of administration typically affecting the choice of solid material. Crystalline solids are characterized by structural periodicity, while amorphous solids lack long-range structural order. The selection of a particular solid may depend on the specific application; amorphous solids are sometimes selected on the basis of, e.g., an enhanced dissolution profile, while crystalline solids may be desirable for properties such as, e.g., physical or chemical stability (see, e.g., S. R. Vippagunta et al., Adv. Drug. Deliv. Rev., (2001) 48:3-26; L. Yu, Adv. Drug. Deliv. Rev., (2001) 48:27-42).

Regardless of whether the solid material is crystalline or amorphous, a pharmaceutical composition comprising a solid form may contain single-component and multiple-component solids. Single-component solids consist essentially of the pharmaceutical compound or active pharmaceutical ingredient without any other compounds. Variability among single-component crystalline materials could potentially develop as a result of polymorphism, a phenomenon characterized by the existence of several three-dimensional crystalline arrangements for a single pharmaceutical compound (see, e.g., S. R. Byrn et al., Solid State Chemistry of Drugs, (1999) SSCI, West Lafayette). Several factors to consider in the design of a crystalline form of a therapeutic agent is that it retains its polymorphic and chemical stability, solubility, and other physiochemical properties over time and among various manufactured batches of the agent. If the physiochemical properties vary with time and among batches, the administration of a therapeutically effective dose becomes problematic and may lead to toxic side effects or to ineffective therapy, particularly if a given polymorph decomposes prior to use, to a less active, or toxic compound. The importance of identifying polymorphs in pharmaceutical compositions was highlighted in the case of Ritonavir™, an HIV protease inhibitor formulated as soft gelatin capsules. Roughly two years after the product launched, a new, less soluble polymorph had been discovered in the formulation, which required the withdrawal of the original capsules from the market and reformulation of the product (see S. R. Chemburkar et al., Org. Process Res. Dev., (2000) 4:413-417).

It is worthwhile to point out that it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.: 3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The existence of solid forms with varying underlying structure creates the possibility to select a solid compound based on its physical and chemical properties for a particular pharmaceutical product. As such, the discovery and choice of solid compounds are of great importance in the development of an effective, stable and marketable pharmaceutical product.

The cannabis plant has many naturally occurring substances. Many substances are available primarily as plant extracts. What is needed, therefore, is solid crystalline forms that can have advantageous properties, including those described herein.

### BRIEF SUMMARY

In certain aspects, the subject matter described herein is directed to crystalline cannabigerol comprising at least one X-ray powder diffraction peak (Cu Kα radiation) selected from 4.73°, 9.52°, 14.30°, and 23.93° 2θ (each ±0.20° 2θ).

In certain aspects, the subject matter described herein is directed to a method of preparing a crystalline cannabigerol composition, comprising:

contacting di-halo olivetol with a geranyl halide in the presence of a base and a first solvent to prepare di-halo cannabigerol;

contacting the di-halo cannabigerol with a reducing agent to prepare a first cannabigerol composition;

contacting said first cannabigerol composition with a second solvent;

and crystallizing a cannabigerol composition having at least one X-ray powder diffraction peak (Cu Kα radiation) selected from 4.73°, 9.52°, 14.30°, and 23.93° 2θ (each ±0.20° 2θ) from said second solvent.

These and other aspects are described fully herein.

### BRIEF DESCRIPTION OF THE FIGURES

FIG. 1A depicts an x-ray powder diffraction pattern of cannabigerol.

FIG. 1B depicts an x-ray powder diffraction pattern of cannabigerol showing peak positions in degrees (Cu Kα radiation).

FIG. 2 depicts a differential scanning calorimetry thermogram of cannabigerol.

FIG. 3 depicts an FT-IR spectrum of cannabigerol.

FIG. 4 depicts a mass spectrum of cannabigerol.

### DETAILED DESCRIPTION

Disclosed herein are novel synthetic routes for the preparation of crystalline cannabigerol. Cannabinoids, such as cannabigerol, may be isolated by extraction or cold pressing from cannabis plants. Isolated compounds from the cannabis plant include Δ⁹-tetrahydrocannabinol (THC), cannabidiol (CBD), cannabichromene (CBC), cannabigerol (CBG), cannabinol (CBN), cannabidivarin (CBDV), among others. While THC has psychoactive effects, CBD, CBC, CBG, and CBDV do not. Cannabinoids have been investigated for possible treatment of seizures, nausea, vomiting, lack of appetite, pain, arthritis, inflammation, and other conditions.

The IUPAC nomenclature for Cannabigerol (CBG) is 2-[(2E)-3,7-dimethylocta-2,6-dienyl]-5-pentyl-benzene-1,3-diol. Its chemical structure is presented below:

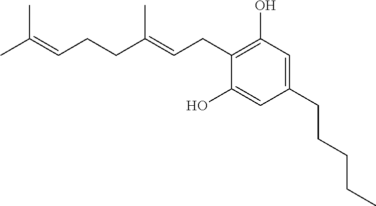 [SMILES: CCCCCc1cc(O)c(C/C=C(\C)CCC=C(C)C)c(O)c1]

CBG is a precursor to the main cannabinoids CBD, CBC, and THC. CBG is found in higher concentrations in hemp plants as opposed to marijuana plants that are grown to contain higher concentrations of tetrahydrocannabinol (THC). CBG has been discovered to act as a high affinity α₂-adrenergic receptor agonist, a moderate affinity 5-HT_{1A} receptor antagonist, and a low affinity CBI receptor antagonist. Although CBG is known to bind with the CB₂ receptor, it is currently unknown whether it acts as an agonist or antagonist.

CBG has been shown to relieve intraocular pressure, which may help in the treatment of glaucoma. It can also be used to treat inflammatory bowel disease. CBG also inhibits the uptake of γ-aminobutyric acid (GABA) in the brain, which in turn helps decrease anxiety. Research on rats has demonstrated that CBG has an anti-nausea and anti-emetic effect, although human testing has yet to be carried out. One study suggested that CBG inhibits the growth of colon cancer cells. Another study found that CBG could potentially be helpful to treat many central nervous system disorders, such as epilepsy.

Over sixty cannabinoids are known to be produced by the cannabis plant. These cannabinoids can be divided among eight main classes: cannabigerol-type; cannabichromene-type; cannabidiol-type; tetrahydrocannabinol-type; cannabielsoin-type; iso-tetrahydrocannabinol-type; cannabicyclol-type; and cannabicitran-type.

All eight classes of cannabinoids are derived from cannabigerol-type compounds and differ predominately in the way the CBG precursor is cyclised. The synthesis of cannabigerol typically begins by loading hexanoyl-CoA onto a polyketide synthase assembly protein and subsequent condensation with three molecules of malonyl-CoA. This polyketide is cyclized to olivetolic acid via olivetolic acid cyclase, and then prenylated with a ten-carbon isoprenoid precursor, geranyl pyrophosphate, using an aromatic prenyltransferase enzyme, geranyl-pyrophosphate—olivetolic acid geranyltransferase, to biosynthesize cannabigerolic acid, which can then be decarboxylated to yield cannabigerol. Cannabigerol is then usually converted by cannabinoid synthase enzymes to cannabidiol (CBD), cannabichromene (CBC) or tetrahydrocannabinol (THC).

The potential therapeutic value of CBG in the treatment of diseases has stimulated research and development in formulating CBG for use in pharmaceutical compositions. CBG is commonly found as an oily plant extract. However, crystalline CBG could be more advantageous in one or more respects compared to other compositions of matter comprising CBG, including amorphous CBG, for example, in terms of chemical and physical stability, storage, processing, compatibility, and hygroscopicity. It is also possible that the crystalline composition could offer easier, quicker, and more extensive dissolution into solvents and more rapidly bioavailability when compared to other forms of CBG. Disclosed herein are unique synthetic routes for obtaining the desired crystalline CBG compositions. While methods for the synthesis of cannabinoids like CBD and THC in the lab environment have been discovered are currently being practiced, synthetic techniques for the preparation of CBG have been relatively unexplored. The presently disclosed novel syntheses use dihalo olivetol and a geranyl halide in the presence of a base and first solvent to generate a dihalocannabigerol intermediate. The intermediate is then contacted with a reducing agent to form a first cannabigerol composition. The first cannabigerol composition is then contacted with a second solvent to generate a crystalline cannabigerol composition. The synthesis proceeds with minimal side reactions to prepare pure, highly crystalline cannabigerol. Evidence of the crystalline form of the CBG obtained from the novel syntheses was obtained through known techniques, including x-ray powder diffraction, FT-IR, and differential scanning calorimetry.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

As used herein, the term “contacting” refers to allowing two or more reagents to contact each other. The contact may or may not be facilitated by mixing, agitating, stirring, and the like.

As used herein, the “CBG” refers to cannabigerol, “CBD” refers to cannabidiol, “CBC” refers to cannabichromene, and “THC” refers to tetrahydrocannabinol.

As used herein, “DBCBD” refers to dibromocannabigerol.

As used herein, “API” refers to Active Pharmaceutical Agent.

As used herein, “FCC” refers to Flash Column Chromatography.

As used herein, “EtOac” refers to ethyl acetate.

As used herein, the terms “halogen,” or “halo” refer to atoms occupying group VIIA of the periodic table, such as fluoro, chloro, bromo or iodo.

As used herein, “plant extract” refers to compositions prepared from solvent extractions from the whole cannabis plant or parts thereof.

As used herein, “substantially free” refers to trace amounts or levels of about 1% w/w or less. As used herein, “essentially free” refers to levels that are below trace. In certain embodiments, essentially free refers to amounts not detectable by standard techniques.

Unless otherwise specified, the term “crystalline” and related terms used herein, when used to describe a substance, component, product, or form, mean that the substance, component, product, or form is substantially crystalline, for example, as determined by X-ray diffraction. (see, e.g., Remington's Pharmaceutical Sciences, 20^{th} ed., Lippincott Williams & Wilkins, Philadelphia Pa., 173 (2000); The United States Pharmacopeia, 37^{th} ed., 503-509 (2014)).

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous forms include, but are not limited to, thermal gravimetric analysis (TGA), differential scanning calorimetry (DSC), X-ray powder diffraction (XRPD), single-crystal X-ray diffraction, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. In the context of molar ratios, “about” and “approximately” indicate that the numeric value or range of values may vary within 20%, 15%, 10%, 9%, 8%, 7%, 6%, 5%, 4%, 3%, 2%, 1.5%, 1%, 0.5%, or 0.25% of the recited value or range of values. It should be understood that the numerical values of the peaks of an X-ray powder diffraction pattern may vary from one machine to another, or from one sample to another, and so the values quoted are not to be construed as absolute, but with an allowable variability, such as ±0.20 degrees two theta (° 20), or more. For example, in some embodiments, the value of an XRPD peak position may vary by up to ±0.20 degrees 2θ while still describing the particular XRPD peak.

As used herein, and unless otherwise specified, a solid form that is “substantially physically pure” is substantially free from other solid forms. In certain embodiments, a crystal form that is substantially physically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other solid forms on a weight basis. The detection of other solid forms can be accomplished by any method apparent to a person of ordinary skill in the art, including, but not limited to, diffraction analysis, thermal analysis, elemental combustion analysis and/or spectroscopic analysis.

As used herein, and unless otherwise specified, a solid form that is “substantially chemically pure” is substantially free from other chemical compounds (i.e., chemical impurities). In certain embodiments, a solid form that is substantially chemically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other chemical compounds on a weight basis. The detection of other chemical compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

As used herein, and unless otherwise indicated, a chemical compound, solid form, or composition that is “substantially free” of another chemical compound, solid form, or composition means that the compound, solid form, or composition contains, in certain embodiments, less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2% 0.1%, 0.05%, or 0.01% by weight of the other compound, solid form, or composition.

Unless otherwise specified, the term “composition” as used herein is intended to encompass a product comprising the specified ingredient(s) (and in the specified amount(s), if indicated), as well as any product which results, directly or indirectly, from combination of the specified ingredient(s) in the specified amount(s). By “pharmaceutically acceptable,” it is meant a diluent, excipient, or carrier in a formulation must be compatible with the other ingredient(s) of the formulation and not deleterious to the recipient thereof.

Unless otherwise specified, to the extent that there is a discrepancy between a depicted chemical structure of a compound provided herein and a chemical name of a compound provided herein, the chemical structure shall control.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound provided herein, with or without other additional active agent, after the onset of symptoms of a particular disease.

A “patient” or “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the patient, individual, or subject is a human.

As used herein, the term “therapeutic amount” refers to an amount of a therapeutic agent, compound, formulation, material, or composition, as described herein effective to achieve a particular biological result. Such results may include, but are not limited to, the inhibition of a disease as determined by any means suitable in the art.

As used herein, the term “pharmaceutically acceptable excipient” refers to an ingredient in a pharmaceutical formulation, other than an active ingredient, which is nontoxic to a subject. A pharmaceutically acceptable excipient includes, but is not limited to, a buffer, carrier, stabilizer, or preservative.

Additional definitions are provided below.

### II. Compositions

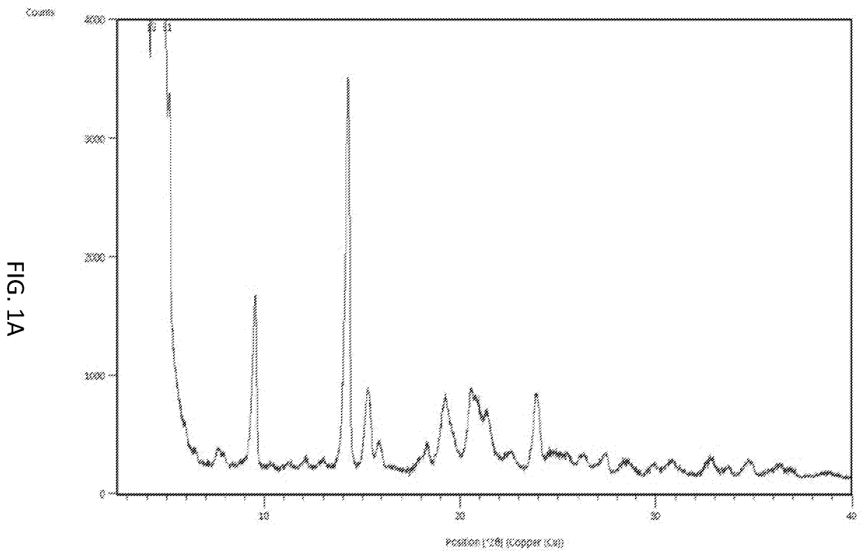

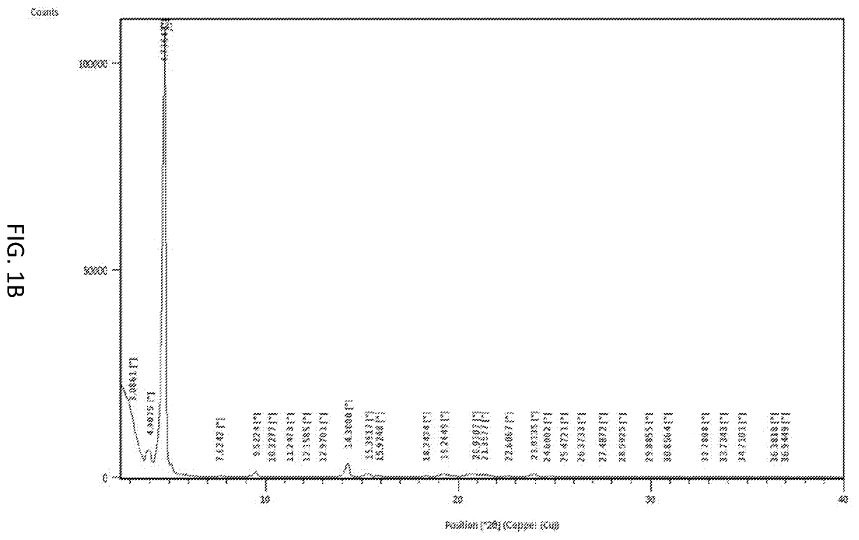

In embodiments, the subject matter described herein is directed to a composition comprising cannabigerol wherein the cannabigerol is a crystalline solid. The crystalline solid has an X-ray powder diffraction pattern substantially as depicted in FIG. 1B. The crystalline solid exhibits a characteristic X-ray powder diffraction pattern with characteristic peaks expressed in 2θ±0.20 at 3.08°, 4.00°, 4.73°, 7.62°, 9.52°, 10.32°, 11.24°, 12.15°, 12.97°, 14.30°, 15.39°, 15.92°, 18.34°, 19.26°, 20.93°, 21.35°, 22.60°, 23.93°, 24.60°, 25.47°, 26.37°, 27.48°, 28.50°, 29.88°, 30.85°, 32.78°, 33.73°, 34.71°, 36.38°, and 36.94°.

In embodiments, the crystalline cannabigerol has at least one X-ray powder diffraction peak selected from the group consisting of 4.73°, 9.52°, 14.30°, and 23.93° 2θ (each ±0.20° 2θ).

In embodiments, the crystalline cannabigerol has at least two X-ray powder diffraction peaks selected from the group consisting of 4.73°, 9.52°, 14.30°, and 23.93° 2θ (each ±0.20° 2θ).

In embodiments, the crystalline cannabigerol has at least three X-ray powder diffraction peaks selected from the group consisting of 4.73°, 9.52°, 14.30°, and 23.93° 2θ (each ±0.20° 2θ).

In embodiments, the crystalline cannabigerol has X-ray powder diffraction peaks at 4.73°, 9.52°, 14.30°, and 23.93° 2θ (each ±0.20° 2θ).

In embodiments, the crystalline cannabigerol has an X-ray powder diffraction pattern obtained using Cu Kα radiation with a wavelength of approximately λ=1.54 Å.

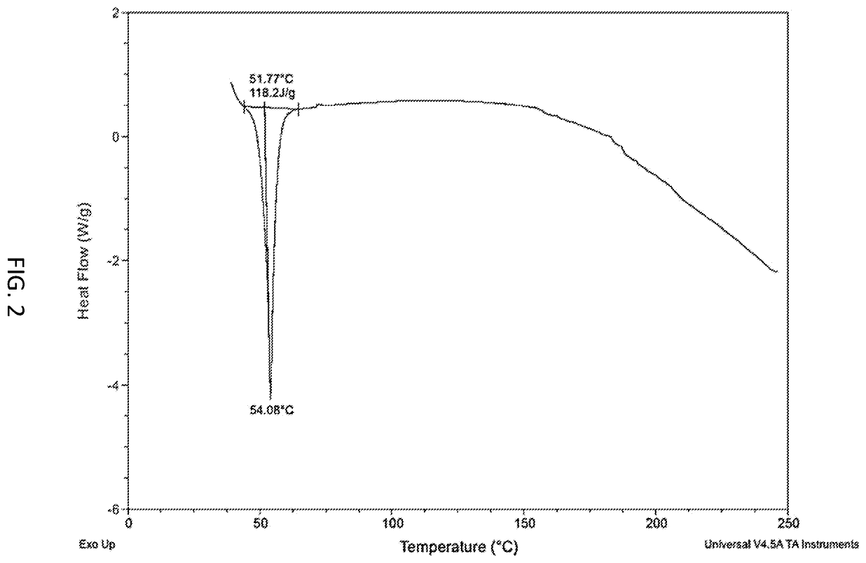

In embodiments, the subject matter described herein is directed to a composition comprising cannabigerol wherein the cannabigerol is a crystalline solid characterized by a differential scanning calorimetry thermogram as set forth in FIG. 2. The cannabigerol is characterized by a differential scanning calorimetry thermogram with an endotherm having an onset at about 51.77° C. and a peak at about 54.08° C.

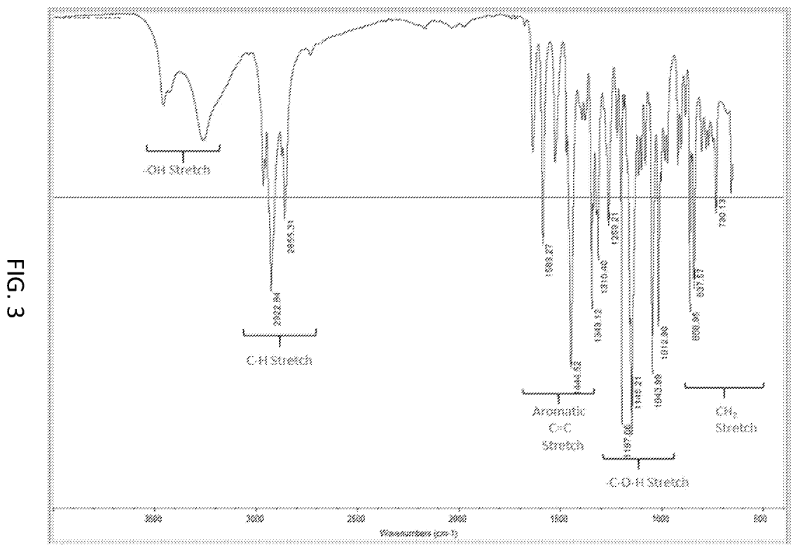

In embodiments, the subject matter described herein is directed to a composition comprising cannabigerol wherein the cannabigerol is a crystalline solid characterized by an IR-FT spectrum as set forth in FIG. 3. The crystalline solid exhibits a characteristic IR-FT spectrum with characteristic peaks expressed at the following positions: about 2922.84 cm⁻¹, about 2855.31 cm⁻¹, about 1583.27 cm⁻¹, about 1444.52 cm⁻¹, about 1343.12 cm⁻¹, about 1310.40 cm⁻¹, about 1259.21 cm⁻¹, about 1197.66 cm⁻¹, about 1145.21 cm⁻¹, about 1043.99 cm⁻¹, about 1012.90 cm⁻¹, about 858.95 cm⁻¹, about 837.57 cm⁻¹, and about 730.13 cm⁻¹ (each ±2.00° cm⁻¹).

In certain embodiments, the X-ray powder diffraction peaks together with the FT-IR and/or the DSC melting point characterizes the crystalline CBG.

Also disclosed are dihalo CBG compounds having the structure:

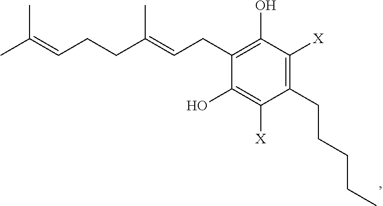

wherein X is a halogen. In certain embodiments, the halogen in each instance is bromo. As disclosed elsewhere herein, synthesis of the crystalline CBG proceeds from a dihalo CBG compound.

### III. Methods

### Methods of Preparation

In certain embodiments, the methods described herein are directed to preparing crystalline cannabigerol in high yield and high purity. Scheme 1 depicts an exemplary route for such a synthesis.

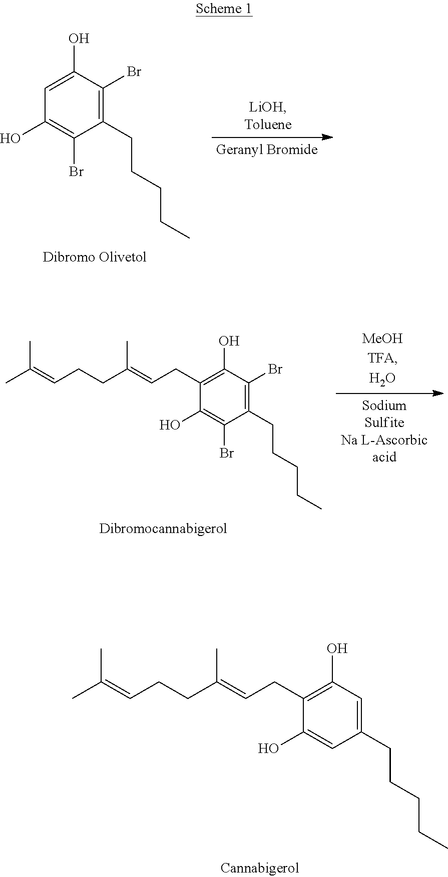

General Procedures

In certain embodiments, olivetol can be substituted with Cl, I, or F, in addition to Br, to form a di-halo Olivetol. Each halogen can be selected from the group consisting of Br, F, I and Cl. In certain embodiments, the halogen is Br.

In certain embodiments, the geranyl halide can be a geranyl bromide, a geranyl chloride, or a geranyl iodide. In certain embodiments, the geranyl halide is a geranyl bromide.

In certain embodiments, di-halo olivetol is contacted with geranyl halide in the presence of a base and a first solvent. In certain embodiments, di-halo olivetol and a base are dissolved in a first solvent prior to contacting the di-halo olivetol with the geranyl halide. Possible bases include LiOH, KOH, NaOH, Sr(OH)₂, Ba(OH)₂, Ca(OH)₂, and RbOH. In certain embodiments, the base is LiOH. In certain embodiments, the first solvent is selected from the group consisting of 2-butanone, ethyl acetate, 1-4-dioxane, diethyl ether, tert-butyl methyl ether, tetrahydrofuran, dichloromethane, chloroform, heptane, toluene, isopropyl acetate, isooctane, n-decane, and anisole. In certain embodiments, the first solvent is toluene.

In certain embodiments, the contacting the di-halo olivetol with geranyl halide is at a temperature of from about 0° C. to about 100° C.; or from about 10° C. to about 90° C.; or from about 20° C. to about 80° C.; or from about 30° C. to about 70° C.; or from about 40° C. to about 60° C.; or from about 45° C. to about 55° C. In certain embodiments, the contacting is at a temperature from about 0° C. to about 20° C., from about 20° C. to about 25° C., from about 25° C. to about 30° C., from about 30° C. to about 35° C., from about 35° C. to about 40° C., from about 40° C. to about 45° C., from about 45° C. to about 50° C., from about 50° C. to about 55° C., from about 55° C. to about 60° C., from about 60° C. to about 65° C., from about 65° C. to about 70° C., about 70° C. to about 75° C., from about 75° C. to about 80° C., or from about 80° C. to about 100° C.

In certain embodiments, the contacting the di-halo olivetol with geranyl halide is for a period of time from about 5 minutes to about 30 hours, or from about 30 minutes to about 20 hours, or from about 1 hour to about 15 hours, or from about 1 hour to about 10 hours, or from about 1.5 hours to about 5 hours. In certain embodiments, the contacting is for a period of time of about 25 hours, about 24 hours, about 23 hours, about 22 hours, about 20 hours, about 17 hours, about 15 hours, about 12 hours, about 10 hours, about 8 hours, about 5 hours, about 2 hours, about 3 hours, about 1 hours, about 15 minutes, about 10 minutes, about 5 minutes, about 10 minutes, about 20 minutes, about 30 minutes, about 45 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, about 5 hours, or more.

In certain embodiments, the contacting the di-halo olivetol with geranyl halide does not occur at a constant temperature. In certain embodiments, the temperature may be raised from about 20° C. to about 60° C., from about 21° C. to about 60° C., from about 22° C. to about 60° C., from about 23° C. to about 60° C., from about 24° C. to about 60° C., from about 25° C. to about 60° C., from about 15° C. to about 50° C., from about 30° C. to about 60° C., from about 35° C. to about 65° C., or from about 25 to about 70° C. In certain embodiments, the contacting the di-halo olivetol with geranyl halide comprises contacting at one temperature for a first period of time, followed by contacting at a second temperature for a second period of time. In certain embodiments, the contacting the di-halo olivetol with geranyl halide takes place at a temperature in a range of about 20-25° C. for about 20 hours, followed by at a temperature of about 60° C. for about 2 hours. In certain embodiments, the contacting the di-halo olivetol with geranyl halide takes place at a first temperature of about 20° C. for about 20 hours, followed by at a second temperature of about 60° C. for about 2 hours. In certain embodiments, the contacting the di-halo olivetol with geranyl halide takes place at a first temperature of about 21° C. for about 20 hours, followed by at a second temperature of about 60° C. for about 2 hours. In certain embodiments, the contacting the di-halo olivetol with geranyl halide takes place at a first temperature of about 22° C. for about 20 hours, followed by at a second temperature of about 60° C. for about 2 hours. In certain embodiments, the contacting the di-halo olivetol with geranyl halide takes place at a first temperature of about 23° C. for about 20 hours, followed by at a second temperature of about 60° C. for about 2 hours. In certain embodiments, the contacting the di-halo olivetol with geranyl halide takes place at a first temperature of about 24° C. for about 20 hours, followed by at a second temperature of about 60° C. for about 2 hours. In certain embodiments, the contacting the di-halo olivetol with geranyl halide takes place at a first temperature of about 25° C. for about 20 hours, followed by at a second temperature of about 60° C. for about 2 hours. In certain embodiments, the second temperature is about 55° C., about 56° C., about 57° C., about 58° C., about 59° C., about 60° C., about 61° C., about 62° C., about 63° C., about 64° C., or about 65° C.

In certain embodiments, after said contacting of the di-halo olivetol with geranyl halide, the reaction mixture is cooled to about 20° C. In certain embodiments, the reaction mixture is cooled to about 15° C., 16° C., 17° C., 18° C., 19° C., 20° C., 21° C., 22° C., 23° C., 24° C., or 25° C. In certain embodiments, cooling the reaction mixture comprises additionally quenching it with a mixture of citric acid and water. In certain embodiments, the mixture of citric acid and water is a 0.87 M solution. In certain embodiments, the mixture is a 0.082 M, 0.083 M, 0.084 M, 0.85 M, 0.86 M, 0.88 M, 0.89 M, or 0.90 M solution.

In certain embodiments, following the cooling and/or quenching of the reaction mixture, the reaction mixture is separated into a top organic layer and a bottom aqueous layer. In embodiments, the organic layer is extracted. In certain embodiments, the organic layer is washed with water. In certain embodiments, the organic layer undergoes evaporation to obtain crude di-halo cannabigerol. In certain embodiments, the crude di-halo cannabigerol is dissolved in a solvent, such as toluene and concentrated to dryness. In certain embodiments, the di-halo cannabigerol is subject to a purification technique, which in some embodiments is column chromatography.

In certain embodiments, the di-halo cannabigerol can then be reduced to remove its halo substituents. The di-halo cannabigerol can undergo reduction by contacting it with a suitably selected reducing agent, for example, sodium sulfite, potassium sulfite, palladium/carbon in combination with hydrogen; in the presence of a suitably selected base, such as sodium hydroxide, triethylamine, sodium carbonate, tripotassium phosphate, and potassium tert-butoxide. In certain embodiments, the base is trimethylamine. The reduction reaction can occur in a suitably selected polar solvent or mixture of polar solvents, or mixture of apolar and polar solvents, for example, methanol or a mixture of methanol and water, acetonitrile, ethanol, acetone, isopropanol, n-butanol, dichloromethane, tetrahydrofuran, tert-butyl methyl ether or a mixture of organic solvent and water. The polar solvent or mixture of polar solvents can also be selected from the group consisting of acetonitrile, methylene chloride, or combinations thereof. In certain embodiments, the solvent is methanol. In certain embodiments, the mixture may also contain a salt of L-ascorbic acid, such as sodium ascorbate.

As used herein, the term “reducing agent” refers to an agent having the ability to add one or more electrons to an atom, ion or molecule. The reducing agent can be a sulfur-containing compound, or Pd/C in the presence of hydrogen. The sulfur containing compound can be a sulfur-containing reducing agent having the ability to reduce C-halogen bonds to form C—H bonds.

The sulfur-containing compound can be a sulfur-containing inorganic acid or salt thereof, including, for example, hydrosulfuric acid (H₂S), sulfurous acid (H₂SO₃), thiosulfurous acid (H₂SO₂O₂), dithionous acid (H₂S₂O₄), disulfurous acid (H₂S₂O₅), dithionic acid (H₂S₂O₂), trithionic acid (H₂S₃O₆) and salts thereof. The sulfur-containing inorganic salt can be an alkali metal salt or an alkaline earth metal salt. For example, the salt can be a monovalent or divalent cation selected from Li⁺, Na⁺, K⁺, Rb⁺, Cs⁺, Fr+, Be²⁺, Mg²⁺, Ca²⁺, Sr²⁺, Ba²⁺, or Ra²⁺. In one embodiment, the salt can be selected from the group consisting of Li⁺, Na⁺, K⁺, Mg²⁺, and Ca²⁺.

The sulfur-containing inorganic salt can also be an ammonium salt (NH₄⁺) or a quaternary ammonium salt. For example, the sulfur-containing inorganic acid salt can be a tetra-alkylated ammonium salt, e.g., a quaternary ammonium salt substituted with four alkyl groups. The alkyl groups can be a C₁-C₁₈. The tetraalkylated ammonium salts can be a tetramethylammonium salt, a tetraethylammonium salt, a tetrapropylammonium salt, a tetrabutylammonium salt, or combinations thereof.

The sulfur-containing inorganic acid or salt thereof can also be one which dissociates into a bisulfite ion (HSO₃⁻) and/or a sulfite ion (SO₃²⁻) in the reaction mixture. Sulfurous acid (H₂SO₃) can generally exist as a solution of SO₂ (commonly about 6%) in water.

In certain embodiments, the reducing agent selected to reduce the di-halo cannabigerol is sodium sulfite.

In certain embodiments, the contacting the di-halo cannabigerol with a reducing agent to prepare a first cannabigerol composition is at a temperature of from about 0° C. to about 100° C.; or from about 10° C. to about 90° C.; or from about 20° C. to about 80° C.; or from about 30° C. to about 70° C.; or from about 35° C. to 75° C., or from about 40° C. to about 60° C.; or from about 45° C. to about 55° C. In certain embodiments, the contacting is at a temperature from about 0° C. to about 20° C., from about 20° C. to about 25° C., from about 25° C. to about 30° C., from about 30° C. to about 35° C., from about 35° C. to about 40° C., from about 40° C. to about 45° C., from about 45° C. to about 50° C., from about 50° C. to about 55° C., from about 55° C. to about 60° C., from about 60° C. to about 65° C., from about 65° C. to about 70° C., from about 70° C. to 80° C., from about 70° C. to about 75° C., from about 75° C. to about 80° C., or from about 80° C. to about 100° C. In certain embodiments, the contacting is at a temperature of about 75° C.

In certain embodiments, the contacting the di-halo cannabigerol with a reducing agent to prepare a first cannabigerol composition is for a period of time from about 5 minutes to about 40 hours, or from about 30 minutes to about 35 hours, or from about 45 minutes to about 20 hours, or from about 1 hour to about 15 hours, or from about 1 hour to about 10 hours, or from about 1.5 hours to about 5 hours. In certain embodiments, the contacting is for a period of time of about 34 hours, about 32 hours, 30 hours, about 28 hours, about 26 hours, about 25 hours, about 24 hours, about 23 hours, about 22 hours, about 20 hours, about 19 hours, about 18 hours, about 17 hours, about 16 hours, about 15 hours, about 12 hours, about 10 hours, about 9 hours, about 8 hours, about 7 hours, about 6 hours, about 5 hours, about 2 hours, about 3 hours, about 1 hours, about 15 minutes, about 10 minutes, about 5 minutes, about 10 minutes, about 20 minutes, about 30 minutes, about 45 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, about 5 hours, or more.

In certain embodiments, after contacting the di-halo cannabigerol with a reducing agent to prepare a first cannabigerol composition, the reaction mixture is concentrated to dryness. In certain embodiments, the reaction mixture is mixed with a solution to an end pH of about 3.7. In certain embodiments, the solution comprises heptane and HCl. In embodiments, a biphasic mixture forms. In certain embodiments, the organic layer is extracted from the aqueous layer. In certain embodiments, the organic layer is washed with a buffer solution comprising K₂HPO₄, KH₂PO₄, sodium ascorbate, and water. In certain embodiments, the organic layer is separated a second time and washed with water. In certain embodiments, the organic layer comprising the first cannabigerol composition is concentrated to dryness a second time.

In certain embodiments, the first cannabigerol composition undergoes purification by a separation technique. In certain embodiments, the separation technique is column chromatography.

In certain embodiments, the first cannabigerol is contacted with a second solvent to obtain crystalline cannabigerol. In certain embodiments, the second solvent is selected from the group consisting of isooctane, chloroform, heptane, dichloromethane, diethyl ether, hexane, n-decane and pentane. In certain embodiments, the second solvent is heptane.

In certain embodiments, the contacting said first cannabigerol composition with the second solvent is at a temperature of about −10° C. In certain embodiments, the contacting temperature is from a temperature of about −30° C. to about 10° C. In certain embodiments, the contacting temperature is from a temperature of about −25° C. to 5° C., −20° C. to 0° C., or from about −15° C. to about −5° C. In certain embodiments, the contacting temperature is at about −14° C., −13° C., −12° C., −11° C., −9° C., −8° C., −7° C., or −6° C.

In certain embodiments, following contacting said first cannabigerol composition with the second solvent to obtain a crystalline cannabigerol composition, the mixture is filtered and washed with more solvent. In certain embodiments, the solvent is heptane. In certain embodiments, the crystalline cannabigerol composition is dried under vacuum at about 30° C. In certain embodiments, the crystalline cannabigerol composition is dried under vacuum at about 25° C., 26° C., 27° C., 28° C., 29° C., 31° C., 32° C., 33° C., 34° C., or 35° C.

In certain embodiments, the amount of base relative to di-halo olivetol is about 0.40 mol to about 0.26 mol (1.5 mol to about 1 mol). In certain embodiments, the amount of base relative to di-halo olivetol is about 0.5 mol to 1 mol, about 0.75 mol to 1 mol, about 1 mol to 1 mol, about 1.25 mol to 1 mol, about 1.75 mol to 1 mol, about 2 mol to 1 mol, about 2.25 mol to 1 mol, about 2.5 mol to 1 mol, about 2.75 mol to 1 mol, about 3 mol to 1 mol.

In certain embodiments, the amount of geranyl halide relative to di-halo olivetol is about 0.32 mol to 0.26 mol (about 1.2 mol to about 1 mol). In certain embodiments, the amount of geranyl halide relative to di-halo olivetol is about 0.6 mol to 1 mol, about 0.7 mol to 1 mol, about 0.8 mol to 1 mol, about 0.9 mol to 1 mol, about 1.1 mol to 1 mol, about 1.3 mol to 1 mol, about 1.4 mol to 1 mol, about 1.5 mol to 1 mol, about 1.6 mol to 1 mol, about 1.7 mol to 1 mol, about 1.8 mol to 1 mol, about 1.9 mol to 1 mol, about 2.0 mol to 1 mol, about 2.25 mol to 1 mol, about 2.5 mol to 1 mol, about 2.75 mol to 1 mol, about 3.0 mol to 1 mol, about 3.5 mol to 1 mol, about 4 mol to 1 mol, or about 5 mol to 1 mol.

The amount of solvent can be adjusted. In certain embodiments, the amount of first solvent relative to di-halo olivetol is about 14.2 mol to about 0.26 mol (about 55 mol to 1 mol). In certain embodiments, the amount of first solvent relative to di-halo olivetol is about 45 mol to 1 mol, about 46 mol to 1 mol, about 47 mol to 1 mol, about 48 mol to 1 mol, about 49 mol to 1 mol, about 50 mol to 1 mol, about 51 mol to 1 mol, about 52 mol to 1 mol, about 53 mol to 1 mol, about 54 mol to 1 mol, about 56 mol to 1 mol, about 57 mol to 1 mol, about 58 mol to 1 mol, about 59 mol to 1 mol, about 60 mol to 1 mol, about 61 mol to 1 mol, about 62 mole to 1 mol, about 63 mol to 1 mol, about 65 mole to 1 mol, about 70 mol to 1 mol, about 75 mol to 1 mol, about 80 mole to 1 mol, about 85 mol to 1 mol, about 90 mole, or about 95 mol to 1 mol.

In certain embodiments, the amount of reducing agent relative to di halo cannabigerol is about 0.55 mol to 1 mol (about 0.06 mol to 0.11 mol). In certain embodiments, the amount of reducing agent relative to di halo cannabigerol is about 0.15 mol to 1 mol, about 0.25 mol to 1 mol, about 0.45 mol to 1 mol, about 0.52 mol to 1 mol, about 0.53 mol to 1 mol, about 0.54 mol to 1 mol, about 0.56 mol to 1 mol, about 0.57 mol to 1 mol, about 0.60 mol to 1 mol, about 0.75 mol to 1 mol, about 0.85 mol to 1 mol, about mol 1 to 1 mol, about 1.15 mol to 1 mol, about 1.25 mol to 1 mol, about 1.5 mol to 1 mol, about 1.75 mol to 1 mol, about 2 mol to about 1 mol, about 1.5 mol to 1 mol, about 2 mol to 1 mol, about 2.5 mol to 1 mol, about 3 mol to 1 mol, about 3.5 mol to 1 mol, about 4 mol to 1 mol, about 4.5 mol to 1 mol, or about 5 mol to 1 mol.

In certain embodiments, the amount of second solvent relative to the first cannabigerol composition is present in a range of about 1 to 1 to about 100 to 1 molar equivalents. In certain embodiments, the amount of second solvent relative to the first cannabigerol composition is present in about 95 to 1, 90 to 1, 80 to 1, 75 to 1, 70 to 1, 65 to 1, 60 to 1, 50 to 1, 45 to 1, 40 to 1, 35 to 1, 30 to 1, 25 to 1, 20 to 1, 15 to 1, 10 to 1, 5 to 1, 4 to 1, 3 to 1, 2 to 1, or 1 to 1 molar equivalents.

In certain embodiments, the present methods attain a crystalline cannabigerol composition in about 5.95% yield. In certain embodiments, the present methods attain a crystalline cannabigerol composition in about 1%, 2%, 3%, 4%, 5%, 5.5%, 5.6%, 5.7%, 5.8%, 5.9%, 5.91%, 5.92%, 5.93%, 5.94%, 5.96%, 5.97%, 5.98%, 5.99%, 6%, 7%, 8%, 9%, 10%, 11%, 12%, 13%, 14%, 15%, 16%, 17%, 18%, 19%, 20%, 25%, 30%, 35%, 50%, 55%, 65%, 60%, 70%, 80%, 90%, 95%, or 99% yield.

In certain embodiments, the compositions described herein comprising cannabigerol further comprise less than 0.15% w/w (E)-4,6-dibromo-2-(3,7-dimethylocta-2,6-dien-1-yl)-5-pentylbenzene-1,3-diol and (E)-4-bromo-2-(3,7-dimethylocta-2,6-dien-1-yl)-5-pentylbenzene-1,3-diol and less than 0.5% 4,6-dibromo Olivetol and 4-bromo-5-pentylbenzene-1,3-diol. In certain embodiments, the composition comprising cannabigerol is substantially free of a halogenated intermediate, such as those listed above. In certain embodiments, the composition comprising cannabigerol is essentially free of a halogenated intermediate.

### Indications and Methods of Treatment

It is contemplated that the crystalline cannabigerol compositions (CBG) disclosed herein may be used to treat a disease. Exemplary diseases include, but are not limited to, emesis, pain, Huntington's disease, Tourette's syndrome, glaucoma, osteoporosis, schizophrenia, cancer, obesity, autoimmune diseases, diabetic complications, infections against methicillin-resistant Staphylococcus aureus, nausea, depression, anxiety, Hypoxia-ischemia injuries, psychosis, and inflammatory diseases.

Autoimmune diseases include, for example, Acquired Immunodeficiency Syndrome (AIDS), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, Parkinson's disease, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjögren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, inflammatory bowel disease, sepsis, stroke, transplantation of tissue and organs, vasculitis, diabetic retinopathy and ventilator induced lung injury.

Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The CBG may be administered by any route appropriate to the condition to be treated, including orally, intravenously, topically, as well as by ophthalmic (eye drops), and transdermal (skin patch) modes.

The CBG can be used either alone or in combination with other agents in a therapy. For instance, the CBG compositions may be co-administered with at least one additional therapeutic agent. Such combination therapies noted above encompass combined administration (where two or more therapeutic agents are included in the same or separate formulations), and separate administration, in which case, administration of the cannabigerol composition can occur prior to, simultaneously, and/or following, administration of the additional therapeutic agent and/or adjuvant.

### IV. Formulations

Pharmaceutical formulations of therapeutic cannabigerol compositions (CBG) as described herein can be prepared for can be prepared for various routes of administration. CBG having the desired degree of purity is optionally mixed with one or more pharmaceutically acceptable excipients (Remington's Pharmaceutical Sciences (1980) 16th edition, Osol, A. Ed.), in the form of a lyophilized formulation for reconstitution or an aqueous solution.

CBG can be formulated in accordance with standard pharmaceutical practice as a pharmaceutical composition. According to this aspect, there is provided a pharmaceutical composition comprising CBG in association with one or more pharmaceutically acceptable excipients. In embodiments, a CBG formulation comprises cannabigerol and a pharmaceutically acceptable excipient.

A typical formulation is prepared by mixing CBG with excipients, such as carriers and/or diluents. Suitable carriers, diluents and other excipients are well known to those skilled in the art and include materials such as carbohydrates, waxes, water soluble and/or swellable polymers, hydrophilic or hydrophobic materials, gelatin, oils, solvents, water and the like. The particular carrier, diluent or other excipient used will depend upon the means and purpose for which the CBG is being applied. Solvents are generally selected based on solvents recognized by persons skilled in the art as safe (GRAS) to be administered to a mammal.

In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEENÔ, PLURONICSÔ or polyethylene glycol (PEG).

The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the CBG or aid in the manufacturing of the pharmaceutical product. The formulations may be prepared using conventional dissolution and mixing procedures.

Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The CBG formulations can be sterile. In particular, formulations to be used for in vivo administration must be sterile. Such sterilization is readily accomplished by filtration through sterile filtration membranes.

The CBG ordinarily can be stored as a solid composition, a lyophilized formulation or as an aqueous solution.

The pharmaceutical compositions comprising CBG can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutic amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

The CBG can be formulated into pharmaceutical dosage forms to provide an easily controllable dosage of the drug and to enable patient compliance with the prescribed regimen. The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The amount of CBG that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The subject matter described herein includes the following embodiments:

1. Crystalline cannabigerol comprising at least one X-ray powder diffraction peak selected from the group consisting of 4.73°, 9.52°, 14.30°, and 23.93° 2θ (each ±0.20° 2θ).
2. The crystalline cannabigerol of embodiment 1, comprising at least two X-ray powder diffraction peaks selected from the group consisting of 4.73°, 9.52°, 14.30°, and 23.93° 2θ (each ±0.20° 2θ).
3. The crystalline cannabigerol of embodiment 1 or 2, comprising at least three X-ray powder diffraction peaks selected from the group consisting of 4.73°, 9.52°, 14.30°, and 23.93° 2θ (each ±0.20° 2θ).
4. The crystalline cannabigerol of any one of embodiments 1-3, comprising X-ray powder diffraction peaks at 4.73°, 9.52°, 14.30°, and 23.93° 2θ (each ±0.20° 2θ).
5. Crystalline cannabigerol of any one of embodiments 1-4 comprising an X-ray powder diffraction pattern substantially similar to that depicted in FIG. 1B.
6. The crystalline cannabigerol of any one of embodiments 1-5, comprising a differential scanning calorimetry thermogram exhibiting an onset at about 51.77° C. and a peak at about 54.08° C.
7. The crystalline cannabigerol of any one of embodiments 1-6, comprising an FT-IR Spectrum comprising a peak at one or more of the following positions: about 2922.84 cm⁻¹, about 2855.31 cm⁻¹, about 1583.27 cm⁻¹, about 1444.52 cm⁻¹, about 1343.12 cm⁻¹, about 1310.40 cm⁻¹, about 1259.21 cm⁻¹, about 1197.66 cm⁻¹, about 1145.21 cm⁻¹, about 1043.99 cm⁻¹, about 1012.90 cm⁻¹, about 858.95 cm⁻¹, about 837.57 cm⁻¹, and about 730.13 cm⁻¹ (each ±2.00° cm⁻¹).
8. A method of preparing crystalline cannabigerol characterized by an X-ray powder diffraction pattern substantially as depicted in FIG. 1B, comprising crystalizing the cannabigerol from heptane.
9. A method of preparing a crystalline cannabigerol composition, comprising:
contacting di-halo olivetol with a geranyl halide in the presence of a base and a first solvent to prepare di-halo cannabigerol;
contacting the di-halo cannabigerol with a reducing agent to prepare a first cannabigerol composition;
contacting said first cannabigerol composition with a second solvent; and
crystallizing from said second solvent a cannabigerol composition comprising at least one X-ray powder diffraction peak selected from the group consisting of 4.73°, 9.52°, 14.30°, and 23.93° 2θ (each ±0.20° 2θ).
   10. The method of embodiment 9, wherein said first solvent is selected from the group consisting of 2-butanone, ethyl acetate, 1-4-dioxane, diethyl ether, tert-butyl methyl ether, tetrahydrofuran, dichloromethane, chloroform, heptane, toluene, isopropyl acetate, isooctane, n-decane, and anisole.
   11. The method of embodiment 9 or 10, wherein said second solvent is selected from the group consisting of isooctane, chloroform, heptane, dichloromethane, diethyl ether, hexane, n-decane and pentane.
   12. The method of any one of embodiments 9-11, wherein prior to said contacting the di-halo olivetol with said geranyl halide, the di-halo olivetol is contacted with said first solvent and said base to form a mixture.

13. The method of any one of embodiments 9-12, wherein said base is selected from the group consisting of LiOH, KOH, NaOH, Sr(OH)₂, Ba(OH)₂, Ca(OH)₂, and RbOH.

14. The method of any one of embodiments 9-13, wherein said reducing agent is a sulfur-containing compound.
15. The method of any one of embodiments 9-14, wherein contacting the di-halo olivetol with said geranyl halide in the presence of said base and said first solvent is at a temperature of from about 20° C. to about 25° C.
16. The method of any one of embodiments 9-15, wherein said contacting takes place for a time period of about 20 hours.
17. The method of any one of embodiments 9-16, wherein contacting the di-halo cannabigerol with said reducing agent is at a temperature in a range of about 70° C. to about 80° C.
18. The method of any one of embodiments 9-17, wherein contacting the di-halo cannabigerol with said reducing agent is at a temperature of about 75° C.
19. The method of any one of embodiments 9-18, wherein said contacting takes place for a time period of between about 18 and 24 hours.
20. The method of any one of embodiments 9-19, wherein contacting said first cannabigerol composition with said second solvent is at a temperature of about −10° C.
21. A pharmaceutical composition comprising the crystalline cannabigerol of any one of embodiments 1-7 and a pharmaceutically acceptable excipient.
22. A method of treating a disease in a subject comprising administering to said subject a composition comprising a therapeutic amount of the crystalline cannabigerol of any one of embodiments 1-7 or a pharmaceutically acceptable salt thereof.
23. The method of embodiment 22, wherein the disease is selected from the group consisting of appetite regulation, obesity, metabolic disorders, cachexia, anorexia, pain, inflammation, neurotoxicity, neurotrauma, stroke, multiple sclerosis, spinal cord injury, Parkinson's disease, levodopa-induced dyskinesia, Huntington's disease, Gilles de la Tourette's syndrome, tardive dyskinesia, dystonia, amyotrophic lateral sclerosis, Alzheimer's disease, epilepsy, schizophrenia, anxiety, depression, insomnia, nausea, emesis, hypertension, circulatory shock, myocardial reperfusion injury, atherosclerosis, asthma, glaucoma, retinopathy, cancer, inflammatory bowel disease, hepatitis, cirrhosis, arthritis, and osteoporosis.

The disclosed subject matter is further described in the following non-limiting Examples. It should be understood that these Examples, while indicating preferred embodiments of the invention, are given by way of illustration only.

### EXAMPLES

### Example 1: Synthesis of Crystalline Cannabigerol

Dibromo Olivetol (0.26 mol), Toluene (14.2 mol), and LiOH (0.4 mol) were charged to a 2000 mL jacketed reactor. Geranyl bromide (0.32 mol) was charged slowly to the stirring mixture at 20-25° C. The reaction mixture was held at 20-25° C. for 20 h. The reaction mixture was heated to 60° C. and held for 2 h. After heating, the mixture was quickly cooled to 20° C. and the reaction was quenched with a mixture of citric acid (0.05 mol) in DI H₂O (3.33 mol). The mixture was separated and the top organic layer was washed with fresh DI H₂O (3.3 mol). The layers were separated and the organic layer was concentrated to dryness via rotary distillation. The crude was dissolved in toluene (1.42 mol) and concentrated to dryness. The crude material was purified via FCC (Heptane:EtOAc, 100:0 to 100:10 over 5 column volumes). The fractions containing dibromocannabigerol were combined and concentrated to dryness.

The crude dibromocannabigerol (0.11 mol) was dissolved in MeOH (12.4 mol) and triethylamine 1.08 mol). This solution was mixed with L-Ascorbic Acid sodium salt (0.03 mol), Sodium Sulfite (0.06 mol) in DI water (27.75 mol). The mixing made a milky mixture. The mixture was heated to 75° C. and held until completion (18 h). The reaction was not complete, and more sodium sulfite was added and the reaction was held for an additional 6 h. Upon reaction completion, the mixture was concentrated to dryness via rotary distillation. The biphasic mixture was mixed with heptane (1.37 mol) and acidified with HCl (0.06 mol) to an end pH of 3.7. The organic layer was separated and then washed with buffer solution (5.5 mol, consisting of K₂HPO₄, KH₂PO₄, sodium ascorbate, in water). The organic layer was separated and washed with DI H₂O (5.5 mol). The organic layer was separated again and concentrated to dryness via rotary distillation. The crude product was purified via FCC (Heptane:EtOAc, 100:0 to 100:10 over 5 column volumes). The fractions containing product were combined and concentrated to dryness.

For crystal formation, the crude was dissolved in heptane (0.25 mol) and cooled to −10° C. The mixture was filtered cold and washed with fresh heptane (0.03 mol). The solids were dried under vacuum at 30° C. Approximately 5.06 g of the cannabigerol product was obtained (5.95% yield).

Characterization Methodology

Samples generated as described in the solid form were analyzed by X-Ray Powder Diffraction (XRPD). XRPD was conducted on a Cubix Pro XRPD (Ser #DY670) using Cu Kα radiation at 1.54 Å. In general, positions of XRPD peaks are expected to individually vary on a measurement-by-measurement basis by about ±0.20° 2θ. In general, as understood in the art, two XRPD patterns match one another if the characteristic peaks of the first pattern are located at approximately the same positions as the characteristic peaks of the second pattern. As understood in the art, determining whether two XRPD patterns match or whether individual peaks in two XRPD patterns match may require consideration of individual variables and parameters such as, but not limited to, preferred orientation, phase impurities, degree of crystallinity, particle size, variation in diffractometer instrument setup, variation in XRPD data collection parameters, and/or variation in XRPD data processing, among others. The determination of whether two patterns match may be performed by eye and/or by computer analysis. An example of an XRPD pattern collected and analyzed using these methods and parameters is provided herein, e.g., as FIG. 1A and FIG. 1B.

Differential Scanning calorimetry (DSC) analyses were performed on a TA Instruments Q200 V24.11. Between 3 and 5 nags of sample was placed into a tared DSC closed aluminum pan and the weight of the sample was accurately recorded. An example of a DSC thermogram collected and analyzed using these methods and parameters is provided herein, e.g., as FIG. 2.

Infrared spectroscopy was performed using a Nicolet iS10 (AKX1401499). Samples were analyzed neat on a smart attenuated total reflectance (ATR) sampling accessory. An example of an IR spectrum collected and analyzed using these methods and parameters is provided herein, e.g., as FIG. 3.

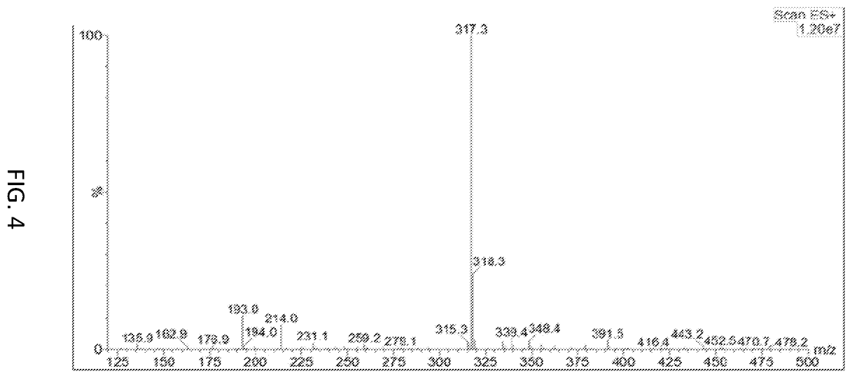

Mass spectroscopy experiments were performed on a Waters Quattro Premier XE MS via direct infusion. An example of a mass spectrum collected and analyzed using these methods and parameters is provided herein, e.g., as FIG. 4.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the disclosure and are encompassed by the appended claims.

Citation or identification of any reference in this application is not an admission that such reference is available as prior art.

Efforts have been made to ensure accuracy with respect to numbers used (e.g. amounts, temperature, etc.) but some experimental errors and deviations should be accounted for.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

The disclosures of all cited references including publications, patents, and patent applications are expressly incorporated herein by reference in their entirety.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller ranges is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation. One skilled in the art will recognize many methods and materials similar or equivalent to those described herein, which could be used in the practicing the subject matter described herein. The present disclosure is in no way limited to just the methods and materials described.

What is claimed is:

1. Crystalline cannabigerol comprising at least one X-ray powder diffraction peak selected from the group consisting of 4.73°, 9.52°, 14.30°, and 23.93° 2θ (each ±0.20° 2θ).

2. The crystalline cannabigerol of claim 1, comprising at least two X-ray powder diffraction peaks selected from the group consisting of 4.73°, 9.52°, 14.30°, and 23.93° 2θ (each ±0.20° 2θ).

3. The crystalline cannabigerol of claim 1, comprising at least three X-ray powder diffraction peaks selected from the group consisting of 4.73°, 9.52°, 14.30°, and 23.93° 2θ (each ±0.20° 2θ).

4. The crystalline cannabigerol of claim 1, comprising X-ray powder diffraction peaks at 4.73°, 9.52°, 14.30°, and 23.93° 2θ (each ±0.20° 2θ).

5. Crystalline cannabigerol comprising an X-ray powder diffraction pattern substantially similar to that depicted in FIG. 1B.

6. The crystalline cannabigerol of claim 1, comprising a differential scanning calorimetry thermogram exhibiting an onset at about 51.77° C. and a peak at about 54.08° C.

7. The crystalline cannabigerol of claim 1, comprising an FT-IR Spectrum comprising a peak at one or more of the following positions: about 2922.84 cm⁻¹, about 2855.31 cm⁻¹, about 1583.27 cm⁻¹, about 1444.52 cm⁻¹, about 1343.12 cm⁻¹, about 1310.40 cm⁻¹, about 1259.21 cm⁻¹, about 1197.66 cm⁻¹, about 1145.21 cm⁻¹, about 1043.99 cm⁻¹, about 1012.90 cm⁻¹, about 858.95 cm⁻¹, about 837.57 cm⁻¹, and about 730.13 cm⁻¹ (each ±2.00° cm⁻¹).

8. A method of preparing crystalline cannabigerol characterized by an X-ray powder diffraction pattern substantially as depicted in FIG. 1B, comprising crystalizing the cannabigerol from heptane.

9. A method of preparing a crystalline cannabigerol composition, comprising:
contacting di-halo olivetol with a geranyl halide in the presence of a base and a first solvent to prepare di-halo cannabigerol;
contacting the di-halo cannabigerol with a reducing agent to prepare a first cannabigerol composition;
contacting said first cannabigerol composition with a second solvent; and
crystallizing from said second solvent a cannabigerol composition comprising at least one X-ray powder diffraction peak selected from the group consisting of 4.73°, 9.52°, 14.30°, and 23.93° 2θ (each ±0.20° 2θ).

10. The method of claim 9, wherein said first solvent is selected from the group consisting of 2-butanone, ethyl acetate, 1-4-dioxane, diethyl ether, tert-butyl methyl ether, tetrahydrofuran, dichloromethane, chloroform, heptane, toluene, isopropyl acetate, isooctane, n-decane, and anisole.

11. The method of claim 9, wherein said second solvent is selected from the group consisting of isooctane, chloroform, heptane, dichloromethane, diethyl ether, hexane, n-decane and pentane.

12. The method of claim 9, wherein prior to contacting the di-halo olivetol with said geranyl halide, the di-halo olivetol is contacted with said first solvent and said base to form a mixture.

13. The method of claim 9, wherein said base is selected from the group consisting of LiOH, KOH, NaOH, Sr(OH)₂, Ba(OH)₂, Ca(OH)₂, and RbOH.

14. The method of claim 9, wherein said reducing agent is a sulfur-containing compound.

15. The method of claim 9, wherein said contacting the di-halo olivetol with said geranyl halide in the presence of said base and said first solvent is at a temperature of from about 20° C. to about 25° C.

16. The method of claim 15, wherein said contacting takes place for a time period of about 20 hours.

17. The method of claim 9, wherein contacting the di-halo cannabigerol with said reducing agent is at a temperature in a range of about 70° C. to about 80° C.

18. The method of claim 17, wherein contacting the di-halo cannabigerol with said reducing agent is at a temperature of about 75° C.

19. The method of claim 18, wherein said contacting takes place for a time period of between about 18 and 24 hours.

20. The method of claim 9, wherein contacting said first cannabigerol composition with said second solvent is at a temperature of about −10° C.

21. A pharmaceutical composition comprising the crystalline cannabigerol of claim 1 and a pharmaceutically acceptable excipient.

22. A method of treating a disease in a subject comprising administering to said subject a composition comprising a therapeutic amount of the crystalline cannabigerol of claim 1 or a pharmaceutically acceptable salt thereof.

23. The method of claim 22, wherein the disease is selected from the group consisting of appetite regulation, obesity, metabolic disorders, cachexia, anorexia, pain, inflammation, neurotoxicity, neurotrauma, stroke, multiple sclerosis, spinal cord injury, Parkinson's disease, levodopa-induced dyskinesia, Huntington's disease, Gilles de la Tourette's syndrome, tardive dyskinesia, dystonia, amyotrophic lateral sclerosis, Alzheimer's disease, epilepsy, schizophrenia, anxiety, depression, insomnia, nausea, emesis, hypertension, circulatory shock, myocardial reperfusion injury, atherosclerosis, asthma, glaucoma, retinopathy, cancer, inflammatory bowel disease, hepatitis, cirrhosis, arthritis, and osteoporosis.
